(19) United States
(12) Patent Application Publication
(10) Pub. No.: US 2017/0014217 A1
(43) Pub. Date: Jan. 19, 2017

(54) INTRAVAGINAL INCONTINENCE DEVICE

(71) Applicant: KAREN L. PATRUSKY, Tenafly, NJ (US)

(72) Inventor: KAREN L. PATRUSKY, Tenafly, NJ (US)

(21) Appl. No.: 14/801,456

(22) Filed: Jul. 16, 2015

(51) Int. Cl. A61F 2/00

(52) U.S. Cl. CPC A61F 2/005

(57) ABSTRACT

An intravaginal incontinence device includes a longitudinal component for insertion into a vagina, wherein the longitudinal component includes a bladder support component configured to provide intravaginal support to a neck of a bladder.

### TECHNICAL FIELD

This disclosure relates to intravaginal devices and, more particularly, to intravaginal incontinence devices.

### BACKGROUND

Urinary incontinence, or the loss of bladder control, is a common problem that impacts millions of people. Further, certain conditions, such as advanced age or childbirth may further exacerbate the problem. With respect to urinary incontinence in women (often as a result of childbirth), various methodologies have be employed. Unfortunately, such methodologies have often concerned absorbing urine upon it release, as opposed to preventing the release of urine.

### SUMMARY OF DISCLOSURE

In one implementation, an intravaginal incontinence device includes a longitudinal component for insertion into a vagina, wherein the longitudinal component includes a bladder support component configured to provide intravaginal support to a neck of a bladder.

One or more of the following features may be included. The bladder support component may include a pair of lateral supports configured to diverge upon insertion into the vagina. The longitudinal component may further include a urethra compression component configured to compress a urethra. The urethra compression component may be configured to diverge from the bladder support component upon insertion into the vagina. The urethra compression component may be configured to apply intravaginal pressure to the urethra. An insertion apparatus may surround the longitudinal component and may be configured to aid in the insertion of the longitudinal component into the vagina. A lubricant may be applied to the longitudinal component. The lubricant may be a water-based lubricant. A removal apparatus may be coupled to the longitudinal component and may be configured to aid in the removal of the longitudinal component from the vagina.

In another implementation, an intravaginal incontinence device includes a longitudinal component for insertion into a vagina, wherein the longitudinal component includes a urethra compression component configured to compress a urethra.

One or more of the following features may be included. The longitudinal component may further include a bladder support component configured to provide intravaginal support to a neck of a bladder. The bladder support component may include a pair of lateral supports configured to diverge upon insertion into the vagina. The urethra compression component may be configured to diverge from the bladder support component upon insertion into the vagina. The urethra compression component may be configured to apply intravaginal pressure to the urethra. An insertion apparatus may surround the longitudinal component and configured to aid in the insertion of the longitudinal component into the vagina. A lubricant may be applied to the longitudinal component. The lubricant may be a water-based lubricant. A removal apparatus may be coupled to the longitudinal component and may be configured to aid in the removal of the longitudinal component from the vagina.

In another implementation, an intravaginal incontinence device includes a longitudinal component for insertion into a vagina, wherein the longitudinal component includes a bladder support component configured to provide intravaginal support to the neck of a bladder and a urethra compression component configured to compress a urethra.

One or more of the following features may be included. The bladder support component may include a pair of lateral supports configured to diverge upon insertion into the vagina. The pair of lateral supports and the urethra compression component may be configured to trifurcate upon insertion into the vagina. The urethra compression component may be configured to diverge from the bladder support component upon insertion into the vagina. The urethra compression component may be configured to apply intravaginal pressure to the urethra. An insertion apparatus may surround the longitudinal component and may be configured to aid in the insertion of the longitudinal component into the vagina. A lubricant may be applied to the longitudinal component. A removal apparatus may be coupled to the longitudinal component and may be configured to aid in the removal of the longitudinal component from the vagina.

The details of one or more implementations are set forth in the accompanying drawings and the description below. Other features and advantages will become apparent from the description, the drawings, and the claims.

### BRIEF DESCRIPTION OF THE DRAWINGS

FIG. 1A is a diagrammatic view of an intravaginal incontinence device in a stored state;

FIG. 1B is a diagrammatic view of the intravaginal incontinence device of FIG. 1A in a deployed state;

FIG. 2A is a diagrammatic view of the intravaginal device of FIG. 1A being inserted for use; and

FIG. 2B is a diagrammatic view of the intravaginal device of FIG. 1A inserted for use.

Like reference symbols in the various drawings indicate like elements.

### DETAILED DESCRIPTION OF THE PREFERRED EMBODIMENTS

### System Overview:

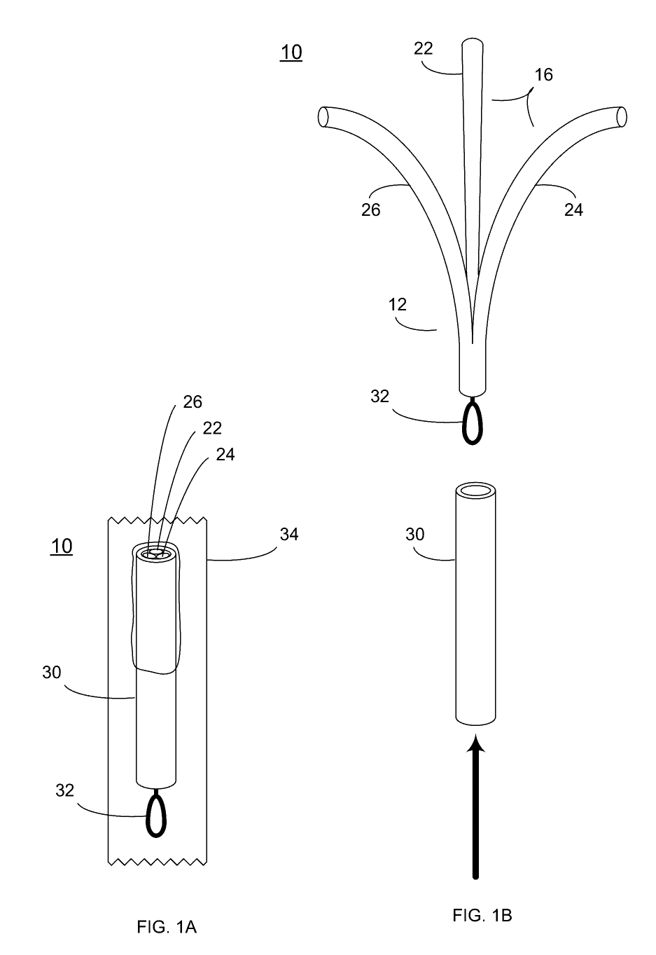

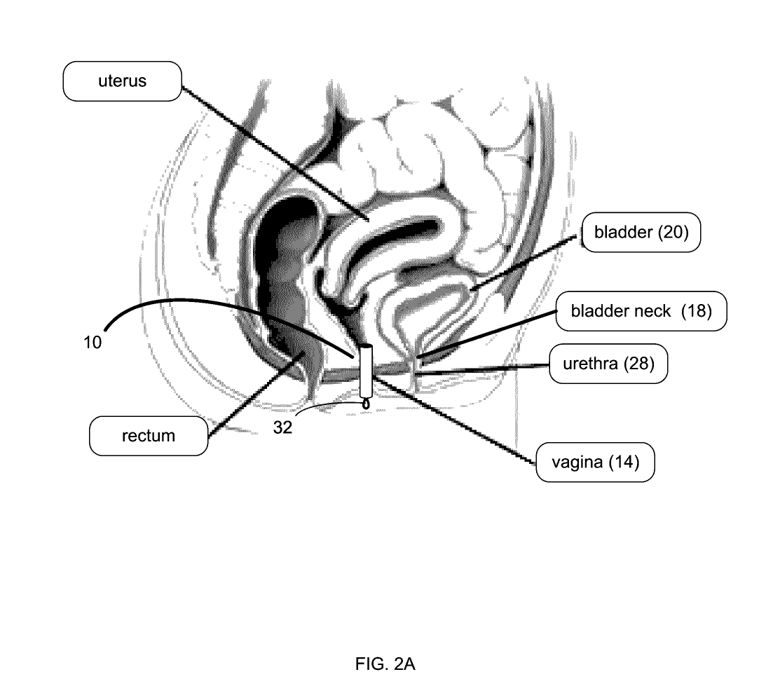

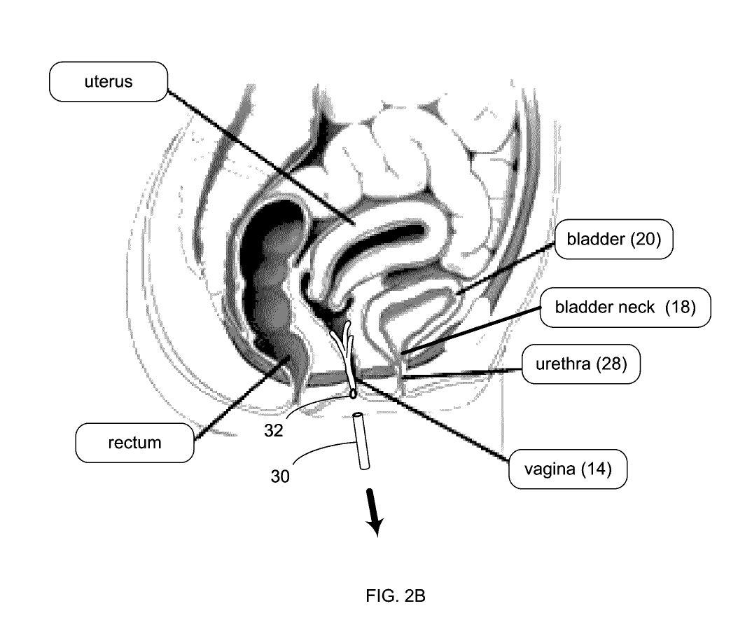

Referring to FIGS. 1A-1B and FIGS. 2A-2B, there is shown intravaginal incontinence device 10. Intravaginal incontinence device 10 may include longitudinal component 12 for insertion into vagina 14. Longitudinal component 10 may include bladder support component 16 configured to provide intravaginal support to neck 18 of bladder 20.

Bladder support component 16 may include two or more lateral supports (e.g., lateral supports 22, 24). For example, bladder support component 16 may include a pair of lateral supports (e.g., lateral supports 22, 24) configured to diverge upon insertion into vagina 14, wherein lateral supports 22, 24 may be configured to provide support to (e.g., lift up) bladder neck 18.

While bladder support component 16 is shown to include two separate and discrete support components (e.g., lateral supports 22, 24), this is for illustrative purposes only and is not intended to be a limitation of this disclosure, as other configurations are possible and are considered to be within the scope of this disclosure. Specifically, the quantity of discrete support components (e.g., lateral supports 22, 24) included within bladder support component 16 may be increased or decreased depending upon design criteria.

Bladder support component 16 (and lateral supports 22, 24) may be constructed of a material that provides the level of resiliency required to e.g., provide support to bladder neck 18 while being compliant enough to be comfortable to the user of intravaginal incontinence device 10. For example, bladder support component 16 (and lateral supports 22, 24) may be constructed of a cotton polymer.

Longitudinal component 12 may further include urethra compression component 26 configured to compress urethra 28. For example, urethra compression component 26 may be configured to apply intravaginal pressure to urethra 28, thus partially (or fully) occluding urethra 28, thus preventing (or reducing) the passage of urine through urethra 28 while intravaginal incontinence device 10 is inserted into vagina 14.

While urethra compression component 26 is shown to include one discrete component, this is for illustrative purposes only and is not intended to be a limitation of this disclosure, as other configurations are possible and are considered to be within the scope of this disclosure. Specifically, the quantity of discrete components included within urethra compression component 26 may be increased or decreased depending upon design criteria.

Urethra compression component 26 may be constructed of a material that provides the level of resiliency required to e.g., to compress urethra 28 while being compliant enough to be comfortable to the user of intravaginal incontinence device 10. For example, urethra compression component 26 may be constructed of a cotton polymer.

Urethra compression component 26 may be configured to diverge from bladder support component 16 upon insertion into vagina 14. Specifically, urethra compression component 26 and lateral supports 22, 24 of bladder support component 16 may be configured to trifurcate (as shown in FIG. 1B) to provide the above-described support to bladder neck 18 and the above-described compression to urethra 28.

Intravaginal incontinence device 10 may include insertion apparatus 30 surrounding longitudinal component 12 that may be configured to aid in the insertion of longitudinal component 12 into vagina 14. Examples of insertion apparatus 30 may include but are not limited to a tube-like structure similar to that of a tampon. During use, intravaginal incontinence device 10 may be inserted into vagina 14 while contained within insertion apparatus 30 (as shown in FIG. 2A) and then insertion apparatus 30 may be removed while allowing longitudinal component 12 to remain inserted into vagina 14 (as shown in FIG. 2B). Once insertion apparatus 30 is removed, urethra compression component 26 and lateral supports 22, 24 of bladder support component 16 may trifurcate (as described above and as shown in FIG. 1B) to provide the above-described support to bladder neck 18 and the above-described compression to urethra 28.

Insertion apparatus 30 may be constructed of various materials that provide the level of rigidity required to e.g., allow for easy insertion of intravaginal incontinence device 10 into vagina 14 while being configured to also provide the desired level of comfort to the user of intravaginal incontinence device 10. For example, insertion apparatus 30 may be constructed of e.g., cardboard or plastic.

Lubricant 32 may be applied to longitudinal component 12 and/or insertion apparatus 30. Specifically and when applied to insertion apparatus 30, lubricant 32 may be configured to aid in the insertion of intravaginal incontinence device 10 into vagina 14. Further and when applied to longitudinal component 12, lubricant 32 may be configured to aid in the removal of intravaginal incontinence device 10 from vagina 14. Examples of lubricant 32 may include but are not limited to a water-based lubricant (e.g., K-Y Jelly).

Intravaginal incontinence device 10 may include removal apparatus 32 coupled to longitudinal component 12, wherein removal apparatus 32 may be configured to aid in the removal of longitudinal component 12 from vagina 14. Removal apparatus 32 may be configured in various manners. For example, removal apparatus 32 may be configured as a loop (as shown in FIGS. 1A-1B and FIGS. 2A-2B) or, alternatively, may be configured as a string (not shown).

Removal apparatus 32 may be constructed of various materials. For example, removal apparatus 32 may be constructed of a non-absorbent material such as a polymer so that removal apparatus 32 does not absorb e.g., any urine that may pass through urethra 28. Alternatively, removal apparatus 32 may be constructed to a more traditional material (e.g., cotton) when absorbency is not a concern.

Since one or more portions of intravaginal incontinence device 10 may be covered with lubricant 32, intravaginal incontinence device 10 may include disposable wrapper 34 that may be impervious to lubricant 32 and may be configured to maintain intravaginal incontinence device 10 in a sterile state.

### General:

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. An intravaginal incontinence device comprising:
a longitudinal component for insertion into a vagina, wherein the longitudinal component includes:
a bladder support component configured to provide intravaginal support to a neck of a bladder.

2. The intravaginal incontinence device of claim 1 wherein the bladder support component includes:
a pair of lateral supports configured to diverge upon insertion into the vagina.

3. The intravaginal incontinence device of claim 1 wherein the longitudinal component further includes:
a urethra compression component configured to compress a urethra.

4. The intravaginal incontinence device of claim 3 wherein the urethra compression component is configured to diverge from the bladder support component upon insertion into the vagina.

5. The intravaginal incontinence device of claim 3 wherein the urethra compression component is configured to apply intravaginal pressure to the urethra.

6. The intravaginal incontinence device of claim 1 further comprising:
an insertion apparatus surrounding the longitudinal component and configured to aid in the insertion of the longitudinal component into the vagina.

7. The intravaginal incontinence device of claim 1 further comprising:
a lubricant that is applied to the longitudinal component.

8. The intravaginal incontinence device of claim 7 wherein the lubricant is a water-based lubricant.

9. The intravaginal incontinence device of claim 1 further comprising:
a removal apparatus coupled to the longitudinal component and configured to aid in the removal of the longitudinal component from the vagina.

10. An intravaginal incontinence device comprising:
a longitudinal component for insertion into a vagina, wherein the longitudinal component includes:
a urethra compression component configured to compress a urethra.

11. The intravaginal incontinence device of claim 10 wherein the longitudinal component further includes:
a bladder support component configured to provide intravaginal support to a neck of a bladder.

12. The intravaginal incontinence device of claim 11 wherein the bladder support component includes:
a pair of lateral supports configured to diverge upon insertion into the vagina.

13. The intravaginal incontinence device of claim 11 wherein the urethra compression component is configured to diverge from the bladder support component upon insertion into the vagina.

14. The intravaginal incontinence device of claim 11 wherein the urethra compression component is configured to apply intravaginal pressure to the urethra.

15. The intravaginal incontinence device of claim 10 further comprising:
an insertion apparatus surrounding the longitudinal component and configured to aid in the insertion of the longitudinal component into the vagina.

16. The intravaginal incontinence device of claim 10 further comprising:
a lubricant that is applied to the longitudinal component.

17. The intravaginal incontinence device of claim 16 wherein the lubricant is a water-based lubricant.

18. The intravaginal incontinence device of claim 10 further comprising:
a removal apparatus coupled to the longitudinal component and configured to aid in the removal of the longitudinal component from the vagina.

19. An intravaginal incontinence device comprising:
a longitudinal component for insertion into a vagina, wherein the longitudinal component includes:
a bladder support component configured to provide intravaginal support to the neck of a bladder, and
a urethra compression component configured to compress a urethra.

20. The intravaginal incontinence device of claim 19 wherein the bladder support component includes:
a pair of lateral supports configured to diverge upon insertion into the vagina.

21. The intravaginal incontinence device of claim 20 wherein the pair of lateral supports and the urethra compression component are configured to trifurcate upon insertion into the vagina.

22. The intravaginal incontinence device of claim 19 wherein the urethra compression component is configured to diverge from the bladder support component upon insertion into the vagina.

23. The intravaginal incontinence device of claim 19 wherein the urethra compression component is configured to apply intravaginal pressure to the urethra.

24. The intravaginal incontinence device of claim 19 further comprising:
an insertion apparatus surrounding the longitudinal component and configured to aid in the insertion of the longitudinal component into the vagina.

25. The intravaginal incontinence device of claim 19 further comprising:
a lubricant that is applied to the longitudinal component.

26. The intravaginal incontinence device of claim 19 further comprising:
a removal apparatus coupled to the longitudinal component and configured to aid in the removal of the longitudinal component from the vagina.
